(19) United States
(12) Patent Application Publication
(10) Pub. No.: US 2021/0378923 A1
(43) Pub. Date: Dec. 9, 2021

(54) NOVEL COMPOSITION

(71) Applicant: GlaxoSmithKline Consumer Healthcare (UK) IP Limited, Brentford, Middlesex (GB)

(72) Inventor: Shazada Yassar KHAN, Weybridge, Surrey (GB)

(21) Appl. No.: 16/771,805

(22) PCT Filed: Dec. 12, 2018
(86) PCT No.: PCT/EP2018/084522

§ 371 Date: Jun. 11, 2020

(30) Foreign Application Priority Data
Dec. 15, 2017 (GB) 1721001.4

(51) Int. Cl. A61K 8/24; A61K 8/73; A61K 8/25; A61K 8/21; A61K 8/34; A61Q 11/00

(52) U.S. Cl. CPC A61K 8/24; A61K 8/731; A61K 8/25; A61K 2800/31; A61K 8/345; A61Q 11/00; A61K 2800/48; A61K 8/21

(57) ABSTRACT

Non-aqueous dentifrice compositions comprising a source of calcium ions and a source of phosphate ions such as a bioactive glass, a humectant such as glycerine, a hydroxyethyl cellulose polymer and a pyrogenic silica. The calcium source and the phosphate source together are precursors for the in situ formation of a desensitizing/remineralizing agent on teeth in the oral cavity. The compositions are useful in remineralizing teeth and in the treatment of dentine hypersensitivity.

### FIELD OF THE INVENTION

The present invention relates to a non-aqueous oral care composition comprising a source of calcium ions and a source of phosphate ions, a humectant, a hydroxyethyl cellulose polymer and a pyrogenic silica. Calcium ions and phosphate ions are essential precursors necessary for the in situ formation of a calcium phosphate-based precipitate, useful in the remineralization of tooth surfaces and in the treatment of dentine hypersensitivity. An example of a source of calcium ions and phosphate ions for use in a composition of the present invention is a bioacceptable and bioactive glass such as a calcium sodium phosphosilicate.

### BACKGROUND OF THE INVENTION

Human tooth enamel—consisting primarily of hydroxyapatite, a crystalline phosphate mineral, naturally undergoes a process of demineralization and remineralization. Saliva, which is supersaturated with respect to calcium and phosphate ions, helps protect teeth against demineralization and can slowly remineralize teeth which have become demineralised by acids. However in today's world of sugary and acidic diets, the natural remineralization process is frequently inadequate to maintain strong enamel. Exposure of saliva and food slowly leaches minerals from teeth and eventually leads to an increased susceptibility to dentine hypersensitivity, dental erosion, caries, incipient caries and even carious dentine demineralization. There has been much work carried out on slowing down the natural process of demineralization and/or of enhancing the process of remineralization, including the development of calcium phosphate-based technologies, with or without fluoride. It is well known that the presence of fluoride ions can enhance the natural remineralization process and this is one of the accepted mechanisms by which fluoride toothpastes serve to strengthen teeth and render tooth enamel more resistant to demineralization.

U.S. Pat. No. 4,080,440 discloses a metastable solution of calcium and phosphate ions at a low pH (between 2.5 and 4) under which conditions the solubility of calcium phosphate salt is high. After penetration of the solution into demineralised enamel, remineralization results from the precipitation of calcium phosphate salts when the pH rises. Flouride ions can be included in the metastable solution. According to U.S. Pat. No. 4,080,440, if remineralization is carried out as contemplated therein, the remineralizaed enamel is more resistant to demineralization than the original enamel. However a significant disadvantage of such metastable solutions is the use of a low pH, potentially resulting in dental enamel demineralization and/or causing injury or irritation to soft oral tissues.

U.S. Pat. No. 4,083,955 discloses a process of remineralization by consecutive treatment of the tooth surface with separate solutions containing calcium ions and phosphate ions. By sequentially and separately applying calcium and phosphate ions, high concentrations of the ions penetrate into the enamel whereby they precipitate as calcium phosphate salts. This method of treatment involves a plurality of sequential applications which are time consuming and inconvenient.

U.S. Pat. No. 5,833,957 discloses an improvement with a two-part system in which calcium and phosphate are kept separate, wherein the two compounds when dispensed are mixed and immediately applied to the teeth for treatment, without the requirement of successive treatments. According to U.S. Pat. No. 5,855,957, the two-part system is necessary to prevent the reaction of the calcium, phosphate and/or fluoride salts. Such a reaction, known to occur in aqueous-based dentifrices, results in the formation of an insoluble calcium phosphate or hydroxyapatite on storage, leading to the unavailability of calcium ions when the dentifrice is in use.

U.S. Pat. No. 4,183,915 discloses a one-part stable aqueous solution comprising calcium ions and phosphate ions for the remimeralization of dental enamel. The solution employs an antinucleating agent to maintain the solubility of calcium phosphate in the presence of fluoride sources.

U.S. Pat. No. 5,866,102 discloses a formulation in the form of a single-part composition comprising a water-soluble calcium salt, a phosphate salt, and a hydrophilic non-aqueous vehicle and optionally a fluoride-releasing agent. To prevent the reaction of the calcium, phosphate and/or fluoride salts, it is necessary for this system to: a) employ a stabilizing desiccating agent; or b) encapsulate or coat the salts with an olephilic or polymeric material which prevents a reaction among the active materials. Although encapsulation is a well known technique that can be usefully employed in the formulation of dentifrice compositions, it does not completely solve the problem as the encapsulated material frequently contacts water due to diffusion or ‘capsule fracture’. It is also more complicated to manufacture as it requires an additional encapsulation or coating step in the manufacturing process.

WO 2002/30381 discloses a composition comprising a non-aqueous carrier, a desensitizing/remineralizing agent consisting essentially of a water-soluble calcium salt, and an incompatible ingredient which would otherwise react with the calcium salt, for reducing dentinal hypersensitivity and remineralizing exposed dentinal surfaces and open dentinal tubules. In one embodiment, the incompatible ingredient is selected from a water-soluble silicate, water-soluble phosphate, and water-soluble fluoride salt, or mixtures thereof. The non-aqueous carrier for a dentifrice composition therein is a single, or a combination of, water-free organic solvents including mineral oils, glycerol, polyol, sorbitol, polyethylene glycol, propylene glycol, copolymers of ethylene oxide and propylene oxide, petrolatum, triacetin and the like. Binders suitable for use include hydroxyethyl cellulose, as well as xanthan gums, Iris moss and gum tragacanth.

WO 1997/27148 discloses a calcium phosphosilicate bioactive glass composition which forms a rapid and continuous reaction with saliva due to the immediate and long-term ionic release of calcium and phosphate to produce a stable crystalline hydroxyapatite layer deposited onto and into dentin tubules for the immediate and long-term reduction of dentin hypersensitivity and tooth surface remineralization.

WO 2009/158564 discloses a method for increasing fluoride uptake onto a tooth structure comprising contacting the tooth structure with a composition that comprises a bioactive glass and fluoride. According to WO 2009/158564 when a bioactive glass is included in a fluoride oral care composition, for example a dentifrice, the release of supplemental calcium and phosphorous from the bioactive glass advantageously increases the uptake of fluoride onto tooth surfaces. The release of these ions can also elicit a modest pH rise that has the potential to increase remineralization in the oral environment. The compositions described therein are non-aqueous compositions for example comprising a polyacrylic acid to thicken a humectant material and to provide the required rheology in order to suspend an abrasive.

According to WO 2010/115037, conventional dentifrice compositions comprising bioactive glass (of the type disclosed in WO 1997/27148) are unsuitable for regular use as toothpastes, because such compositions are water-based and the calcium ions released by the bioactive glass reacts and crosslinks with water molecules to form unacceptably thick pastes. According to WO 2010/115037, non-aqueous dentifrice compositions comprising a gum selected from the group consisting of carrageenean and carboxymethylcellulose, at least one humectant and a bioactive glass, provide dentifrices that are suitable for routine, regular use and exhibit acceptable mouth-feel, foam and product stability.

As may be seen from the prior art cited hereinabove, use of non-aqueous (anhydrous) carrier or vehicle systems is generally known in the art. Such systems have been suggested as a means of overcoming incompatibility or stability problems associated with use of aqueous-based dentifrice compositions.

U.S. Pat. No. 5,670,137 describes an anhydrous dentifrice composition based on glycerine, hydroxyethylcellulose with a hydrophobic chain, and a pyrogenetic silica for use in bucco-dental hygiene. According to U.S. Pat. No. 5,670,137, the compositions therein permit the introduction of active agents that are slightly stable or unstable in aqueous medium and which, in use, exhibit smoothness, homogeneity, bright characteristics, viscosity, consistency and cleaning and polishing capacity.

WO 2002/38119 describes a non-aqueous dentifrice composition suitable as a vehicle for materials that are incompatible with an aqueous environment. The composition comprises a hydroxyethyl cellulose polymer, a humectant, a polyethylene glycol and a dentally acceptable abrasive.

It has now been discovered that a non-aqueous composition that comprises a humectant, a hydroxyethyl cellulose polymer and a pyrogenic silica facilitates the delivery of calcium ions and phosphate ions to tooth surfaces and enhances the formation of a calcium phosphate desensitizing/remineralizing precipitate. The composition is suitable for routine, regular use and ideally will provide one or more properties that are key drivers of consumer acceptance such as acceptable taste, consistency and adequate foaming on brushing of teeth.

### SUMMARY OF THE INVENTION

The present invention relates to a non-aqueous oral care composition comprising a source of calcium ions, a source of phosphate ions, a humectant, a hydroxyethyl cellulose polymer and a pyrogenic silica.

The invention further provides a method for desensitizing hypersensitive teeth by applying thereto a desensitizing amount of a non-aqueous oral care composition comprising a calcium ion source and a phosphate ion source, a humectant, a hydroxyethyl cellulose polymer and a pyrogenic silica.

### DETAILED DESCRIPTION OF THE INVENTION

### Brief Description of the Drawings

FIG. 1—shows a QCM-D with lid schematic;

FIG. 2—shows precipitate deposition at initial and two hour time points for various formulations, as determined using the QCM-D equipment shown in FIG. 1;

FIG. 3—shows Hydraulic Conductance (HC) data for various formulations.

As used herein the word “comprising” includes its normal meaning (i.e. includes all the specifically mentioned features as well optional, additional, or unspecified ones), and also includes “consisting of” and “consisting essentially of”.

As used herein, the word “about”, when applied to a value for a parameter of a composition indicates that the calculation or measurement of the value allows some slight imprecision without having a substantial effect on the chemical or physical attributes of the composition.

As used herein the term “desensitizing amount” means considering the method of delivery and formulation, an amount that is sufficient to aid in desensitizing sensitive teeth.

Suitably the oral care composition of the present invention is in the form of a semi-solid such as a dentifrice or balm. In one embodiment the oral care composition is in the form of a dentifrice. Suitably the dentifrice is in the form of an extrudable semi-solid such as a cream, paste or gel (or mixture thereof).

The oral care composition of the invention is a product that in the ordinary course of usage is retained in the oral cavity for a time sufficient to contact some or all of the surfaces of the teeth for purposes of oral activity, including the in situ generation of a calcium phosphate-based desensitizing/remineralizing precipitate.

The present invention is based on the unexpected finding that a non-aqueous oral care composition according to the invention provides enhanced deposition of a desensitizing/remineralizing precipitate based on calcium and phosphate, on a tooth surface. Up to four times more precipitate deposition was observed with a non-aqueous composition according to the invention comprising a bioactive glass as a source of calcium ions and of phosphate ions, a hydroxyethyl cellulose polymer (as a thickening agent) and a pyrogenic silica (as a thickening agent), as compared to a control composition i.e. a non-aqueous composition comprising a bioactive glass as a source of calcium ions and of phosphate ions, a polyacrylic acid (as a thickening agent) and a conventional thickening silica. Whilst not being bound by theory, it is believed that the hydroxyethyl cellulose polymer and the pyrogenic silica, present in the composition, facilitate the release of calcium ions and phosphate ions and further serve to promote the formation of a desensitizing/remineralizing precipitate. In contrast to polyacrylic acid, the hydroxyethyl cellulose polymer of use in the present invention does not appear to interfere with or hinder precipitate formation. Pyrogenic silica, in contrast to conventional thickening silica, is believed to provide additional nucleation sites that further facilitate the formation of the desensitizing/remineralizing precipitate.

As a consequence of any enhanced or improved precipitate formation, a composition according to the invention may exhibit improved remineralization properties thereby reducing further the likelihood of dentine hypersensitivity, dental erosion, caries, and/or may result in improved appearance of teeth by whitening through generation of new hydroxyapatite or hydroxyapatite-like material.

An oral care dentifrice composition according to the invention exhibits acceptable physical stability and structure and does not exhibit a runny character, despite its non-aqueous nature. The composition is cost-effective and easy to manufacture.

Advantageously a composition according to the invention is in the form of a single phase composition. There is no requirement to keep the calcium ion source and phosphate ion source separate from one another in order to avoid any premature reaction between the two sources. This is in contrast to prior art compositions where calcium and phosphate sources are kept apart until just prior to use, for example as seen with the dual phase compositions as disclosed in WO 2012/143220.

These and other features, aspects and advantages of the invention will become evident to those of skill in the art from a reading of the present disclosure.

An oral care composition of the present invention is non-aqueous i.e. is substantially free of any water. This is achieved by not adding water to the composition, by not using an aqueous carrier(s) and, where possible, by avoiding use of components in their hydrated form. Suitably a component selected for use in the composition will be in its anhydrous form. Whilst recognizing that individual components of the composition may contain limited amounts of free and/or bound water, it is essential that the overall composition remains substantially free of any water. Aqueous carriers of the type commonly used in dentifrice compositions are avoided in the present invention; these include for example aqueous solutions of sodium lauryl sulphate, aqueous solutions of sodium hydroxide and aqueous solutions of colouring agents. The total amount of water (both free and bound water) in a composition of the invention is kept to a minimum. Suitably a composition of the invention will comprise less than 5% water by weight of the composition, suitably less than 3% water by weight of the composition, and even more suitably less than 1% water by weight of the composition.

It will be recognized by those skilled in the art that different types of a calcium phosphate-based desensitizing/remineralizing precipitate can be formed during use, by a composition according to the present invention. The desensitizing/remineralizing precipitate formed will depend upon the calcium ion source and the phosphate ion source used in the composition. Suitably the precipitate formed includes hydroxyapatite e.g. represented by the formula Ca₁₀(PO₄)₆(OH)₂, calcium silicate, fluoroapatite e.g. represented by the formula (Ca₁₀(PO₄)6F₂), a tricalcium phosphate e.g. represented by the formula (Ca₁₀(PO₄)₂), and various other kinds of known calcium phosphate-based compounds depending upon the calcium and phosphate sources and other ingredients, such as fluoride, present in the composition.

In one aspect the desensitizing/remineralizing precipitate formed is a hydroxyapatite, hydroxycarbonate apatite, calcium silicate, fluoroapatite, tricalcium phosphate or mixtures thereof.

In one embodiment the desensitizing/remineralizing precipitate formed is hydroxycarbonate apatite.

A composition according to the invention comprises a source of calcium ions and a source of phosphate ions. In one embodiment the calcium ions and the phosphate ions are from the same source i.e. a compound containing both calcium and phosphate (hereinafter referred to as the “calcium phosphate compound”).

Suitably the calcium phosphate compound may be selected from the group consisting of a bioactive glass, calcium glycerophosphate, dicalcium phosphate dihydrate, tetracalcium phosphate, octacalcium phosphate, amorphous calcium phosphate, apatite, α-tricalcium phosphate or a mixture thereof.

In one embodiment the calcium phosphate compound may be selected from the group consisting of a bioactive glass, apatite, calcium glycerophosphate or a dicalcium phosphate dihydrate or a mixture thereof.

In one embodiment the calcium phosphate compound is a bioacceptable and bioactive glass.

In one embodiment the bioacceptable and bioactive glass is calcium sodium phosphosilicate.

A bioactive glass for use in the present invention typically is formed from a combination of silicon dioxide (SiO₂), calcium oxide (CaO), sodium oxide (Na₂O) and phosphorous oxide (P₂O₅) wherein one or more of the preceding oxides may be replaced by one of more of the following: Strontium oxide (SrO); boron trioxide (B₂O₃); potassium oxide (K₂O); magnesium oxide (MgO); zinc oxide (ZnO); MFₓ where M is a monovalent or divalent cation and x is 1 or 2.

In one embodiment the bioactive glass is formed from a combination of 40% to 60% by weight silicon dioxide, from 10% to 40% by weight calcium oxide, from 10% to 35% by weight sodium oxide, from 2% to 8% phosphorus oxide, from 0% to 25% by weight calcium fluoride, from 0% to 10% by weight boron oxide, from 0% to 8% by weight potassium oxide, from 0% to 5% magnesium oxide.

In a further embodiment the bioactive glass comprises about 45% by weight silicon dioxide, about 24.5% by weight sodium oxide, about 6% by weight phosphorus oxide, and about 24.5% by weight calcium oxide. In one such embodiment, the bioactive glass is a calcium sodium phosphosilicate bioactive glass available commercially under the trade name, NovaMin®, also known as 45S5 Bioglass®.

Without being bound by theory, it is believed that upon contact with saliva, sodium ions (Na⁺) present in calcium sodium phosphosilicate bioactive glass particles begin to exchange rapidly with H⁺ present in the saliva. This exchange allows calcium (Ca²⁺) and phosphate (PO₄³⁻) species to be released from the particle structure. A modest, localized, transient increase in pH occurs that facilitates the precipitation of calcium and phosphate from the particles and from saliva to form a calcium-phosphate (Ca—P) layer on tooth surfaces. As the reactions and deposition of Ca—P complexes continue, a crystalline hydroxycarbonate apatite (HCA) layer forms that is structurally and chemically similar to natural tooth mineral.

A bioactive glass for use in an oral composition of the present invention is in particulate form and has an average particle size, (as determined by laser diffraction), less than or equal to about 500 μm, suitably less than about 250 μm or less than about 150 μm. In some embodiments of the present invention, small particles are used; for example particles having an average particle size of less than 100 μm, such as in the range of about 0.01 μm to about 90 μm or about 0.1 μm to about 25 μm.

Suitably the calcium phosphate compound is present in a composition of the invention in an amount ranging from 0.5 to 20% by weight of the composition, more suitably from 1 to 10% by weight of the composition.

In one embodiment the calcium ions and the phosphate ions are from different sources. The calcium ion source includes any toxicologically harmless calcium compound that is capable of reacting with a source of phosphate ions to form a desensitizing/remineralizing precipitate in situ upon contact with saliva in the mouth.

Suitable calcium sources that may be used in this context include, for example: calcium chloride, calcium bromide, calcium nitrate, calcium acetate, calcium gluconate, calcium benzoate, calcium glycerophosphate, calcium formate, calcium fumarate, calcium lactate, calcium butyrate and calcium isobutyrate, calcium malate, calcium maleate, calcium tartrate, calcium succinate, calcium propionate, calcium carbonate, calcium silicate, calcium oxide, calcium sulphate, calcium alginate or mixtures thereof.

In one embodiment the calcium ion source is selected from calcium silicate, calcium carbonate, calcium sulphate and mixtures thereof.

When a calcium silicate is employed, the same may comprise calcium oxide-silica (CaO—SiO₂) as described in PCT applications published as WO 2008/015117 and WO 2008/068248.

When a calcium sulphate is employed, the same may comprise anhydrous calcium sulphate, calcium sulphate hemihydrate and calcium sulphate dihydrate as described in U.S. Pat. No. 6,159,448.

Suitably the amount of calcium ion source in a composition of the invention ranges from 0.5 to 20% by weight of the composition, more suitably from 1 to 10% by weight of the composition.

The phosphate ion source employed in a composition of the invention includes any toxicologically harmless phosphate compound that is capable of reacting with a calcium source to form a desensitizing/remineralizing precipitate in situ upon contact with saliva in the mouth.

Suitable phosphate ion sources that may be used in this context include, for example: sodium dihydrogen phosphate, disodium hydrogen phosphate, sodium pyrophosphate, tetrasodium pyrophosphate, sodium tripolyphosphate, sodium hexametaphosphate, potassium dihydrogenphosphate, trisodium phosphate, tripotassium phosphate or mixtures thereof.

In one embodiment the phosphate ion source is a mixture of trisodium phosphate and sodium dihydrogen phosphate.

In one embodiment the calcium ion source is a calcium silicate and the phosphate ion source is a mixture of trisodium phosphate and monosodium dihydrogen phosphate.

Suitably the amount of phosphate ion source(s) in a composition of the invention ranges from 0.5 to 20% by weight of the composition, more suitably from 1 to 10% by weight of the composition.

A composition according to the invention comprises a humectant. Suitable humectants for use in the present invention include glycerine, sorbitol and propylene glycol or mixtures thereof. In one embodiment the humectant is glycerine. It is well known that commercially available glycerine may contain between about 0.5 to about 2.0% by weight of water which is in association with the glycerine. Typically this amount is between about 0.5 to about 1.0% by weight. This small amount of water is bound to the glycerine and is therefore not available to the other ingredients. The skilled person would still consider a composition containing glycerine as being non-aqueous. The humectant should in any case be as anhydrous as possible and preferably used in solid form. As the humectant is used to make the formulations up to 100%, the humectant may be present in the range of from about 20% to about 95% by weight of the composition. Suitably the humectant is present from about 50% to about 90% by weight of the composition. In one embodiment the humectant is present from about 70% to about 96% by weight of the composition.

A composition according to the invention further comprises a hydroxyethyl cellulose polymer and a pyrogenic silica, which serve as thickening agents in the composition. Thickening agents are required to bind the ingredients of the composition together and to impart adequate texture and rheology during preparation, storage and utilisation. Advantageously the thickening agents of use herein facilitate the in situ formation of the desensitizing/remineralizing precipitate.

In one aspect a composition according to the invention is essentially free of any further/additional thickening agent(s).

In one aspect a composition according to the invention is essentially free of a polyacrylic acid.

A suitable hydroxyethyl cellulose polymer of use in an oral care composition of the invention includes a high, medium and low viscosity grade with differing levels of ethylene oxide substitution. A hydroxyethyl cellulose polymer of use in the invention is one that has not been modified by the introduction of a hydrophobic alkyl or aralkyl group. This is in contrast with the hydroxyethyl cellulose polymer disclosed for use in the dentifrice compositions of U.S. Pat. No. 5,670,137, which is modified and comprises a hydrophobic chain.

Accordingly in one aspect a composition according to the invention is free or essentially free of a hydroxyethylcellulose polymer which has been modified by the introduction of a hydrophobic alkyl or aralkyl group. By “essentially free” is meant that the compositions have no more than 0.01% by weight of these modified polymers.

In one embodiment the hydroxyethyl cellulose polymer has a particle size range of between 5 and 800 micrometers, such as between 10 and 250 micrometers. In one embodiment the hydroxyethyl cellulose has a viscosity (when measured as a 1% w/w aqueous solution at 25° C.) of between 100 and 6000 mPa·s.

Suitably a hydoxyethyl cellulose polymer for use in the invention is available commercially under the trade name Natrosol. Examples of such polymers include the following with the below indicated properties:

| | Average | Brookfield | |
|---|---|---|---|
| | molecular | LVF viscosity | Solution |
| | weight | at 25° C., | concentration |
| Grade | (Da) | mPa s | (%) |
| Natrosol 250 L pharm | 90,000 | 75-150 | 5 |
| Natrosol 250 G pharm | 300,000 | 250-400 | 2 |
| Natrosol 250 M pharm | 720,000 | 4,500-6,500 | 2 |
| Natrosol 250 H pharm | 1,000,000 | 1,500-2,500 | 1 |
| Natrosol 250 HHX pharm | 1,300,000 | 3,500-5,500 | 1 |

A hydroxyethyl cellulose polymer suitable for use in the present invention, is Natrosol MX available commercially from Hercules Inc, Aqualon Division, Hercules Plaza, 1313 North Market Street, Wilmington, Del. 19894-0001. Natrosol MX exhibits a viscosity (when measured as a 2% w/w aqueous solution at 25° C., using a Brookfield LVF having a spindle number 4 and an RPM of 60) of 4,500-6,500 mPa·s.

Suitably the hydroxyethyl cellulose polymer may be present in the range of from 0.1% to 7.5% by weight of the composition, suitably from 0.3% to 2.0%.

A composition according to the invention comprises a pyrogenic silica, as a thickening silica. Pyrogenic silica (also known as fumed silica) is a form of synthetic, amorphous silica, and usually is prepared from SiCl₄ in a flame. Pyrogenic silica is a fluffy white powder consisting of microscopic droplets of amorphous silica, fused into branched, chain-like three dimensional particles which then agglomerate into tertiary particles. Pyrogenic silica of use in the invention is essentially non-porous and has a BET surface area in the range of about 50-600 m²/g.

Suitably the pyrogenic silicas of use in the invention have an average primary particle size of less than 40 nm, more suitably not more than 30 nm. The average primary particle size is suitably between 5 and 30 nm.

The pyrogenic silicas of use in the invention are hydrophilic. Among the hydrophilic pyrogenic silicas which have an average particle size of less than 40 nm are the products marketed under the names Aerosil 90, Aerosil 130, Aerosil 150, Aerosil 200, Aerosil 300 and Aerosil 380 by the Degussa Company.

Suitably the pyrogenic silica may be present in the range of from 0.1% to 10% by weight of the composition, suitably from 0.3% to 5.0%.

Suitably a composition according to the invention comprises an abrasive silica. Generally, an amount of abrasive suitable for use in the composition of the present invention will be empirically determined to provide an acceptable level of cleaning and polishing, in accordance with the techniques well known in the art. Suitably, the abrasive will be present in an amount from about 1% to about 60% by weight of the composition, suitably from about 2% to about 30% by weight of the composition or from about 3% to about 10%, by weight of the composition.

Surfactant materials are usually added to dentifrice products to provide cleaning and/or foaming properties. Any conventional surfactant used in dentifrice formulations may be used in the present invention, provided that it can be added as a solid powder that is not in an aqueous solution.

Suitable surfactants include anionic, cationic, nonionic and amphoteric surfactants.

Suitable nonionic surfactants include, for example polyethoxylated sorbitol esters, in particular polyethoxylated sorbitol monoesters, for instance, PEG(40) sorbitan diisostearate, and the products marketed under the trade name ‘Tween’ by ICI; polycondensates of ethylene oxide and propylene oxide (poloxamers), for instance the products marketed under the trade name ‘Pluronic’ by BASF-Wyandotte; condensates of propylene glycol; polyethoxylated hydrogenated castor oil, for instance, cremophors; and sorbitan fatty esters.

Suitable anionic surfactants include, for example sodium lauryl sulphate, marketed by Albright and Wilson and known as ‘SLS’. When used in the present invention, SLS is used in powder form. A further suitable anionic surfactant is sodium methyl cocyl taurate, marketed under the trade name ‘Adinol CT 95’ manufactured by Croda chemicals.

Suitable amphoteric surfactants include, for example a betaine. Structurally, betaine compounds contain an anionic functional group such as a carboxylate functional group and a cationic functional group such as quaternary nitrogen functional group separated by a methylene moiety. They include n-alkyl betaines such as cetyl betaine and behenyl betaine, and n-alkylamido betaines such as cocoamidopropyl betaine. In one embodiment the betaine is cocoamidopropyl betaine, commercially available under the trade name Tego Betain.

Advantageously, the surfactant is present in an amount ranging from about 0.005% to about 20% by weight of the composition, suitably from about 0.1% to about 10% by weight of the composition, more suitably 0.1% to 5% by weight of the composition.

Advantageously a composition according to the invention may further comprise an ionic fluorine-containing compound, which may include ionic fluorides, such as alkali metal fluorides, amine fluorides and ionic monofluorophosphates, such as alkali metal monofluorophosphates, and which may be incorporated into the formulation, to provide between 100 and 3000 ppm, preferably 500 to 2000 ppm of fluoride. Preferably the ionic fluoride or monofluorophosphate is an alkali metal fluoride or monofluorophosphate, for instance sodium fluoride or sodium monofluorophosphate, respectively. It will further be appreciated that if an ionic fluoride-containing compound is incorporated in a composition of the invention, the abrasive should be chosen so that it is compatible with the ionic fluorine-containing compound.

Compositions of the present invention may further comprise one or more active agents conventionally used in oral healthcare compositions, for example, a desensitising agent, an anti-erosion agent, an anti-plaque agent, an anti-calculus agent, a whitening agent, a breath freshening agent and a tooth whitening agent. Such agents may be included at levels to provide the desired therapeutic effect.

Compositions of the present invention may comprise a desensitising agent, for combating dentine hypersensitivity. Examples of desensitising agents include a tubule blocking agent or a nerve desensitising agent and mixtures thereof, for example as described in WO 02/15809. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

A desensitising amount of a potassium salt is generally between 2 to 8% by weight of the total composition, for example 5% by weight of potassium nitrate can be used.

Compositions of the present invention may comprise an anti-erosion agent, for example a polymeric mineral surface active agent or a stannous, zinc or copper compound, as described in WO 04/054529 (Procter & Gamble) or a nanoparticulate zinc oxide, as described in WO 08/054045 (Glaxo Group Limited), or a mixture thereof.

Suitable anti-plaque agents for use in a composition according to the invention include triclosan, chlorhexidine or cetyl pyridnium chloride. Suitable anti-calculus agents include pyrophosphate salts. A suitable breath freshening agent includes sodium bicarbonate. Suitable tooth whitening agents include hydrogen peroxide and sodium tripolyphosphate.

A composition according to the invention may also contain other agents conventionally used in oral health formulations, for example colouring agents, preservatives, flavouring agents and sweetening agents.

In general, such agents will be in a minor amount or proportion of the composition, usually present in an amount ranging from about 0.001% to about 5% by weight of the composition. Because of the inventive combination of ingredients used in the present invention, any active ingredient or combination of actives that are unstable or incompatible in any way with aqueous environments may also be added to the composition of the present invention. Flavouring agents may be added to the compositions, usually at a typical level of about 1.0% by weight of the composition.

Suitable sweetening agents include saccharin, cyclamate and acesulfame K, and may be present in from about 0.01% to about 0.5%, suitably from about 0.05% to about 0.5% by weight of the composition. An auxiliary sweetener such as a thaumatin may also be included, at a level of from about 0.001% to about 0.1%, suitably from about 0.005% to about 0.05% by weight of the composition. A suitable blend of thaumatins is marketed under the trade name ‘TALIN’ by Tate and Lyle plc.

A composition according to the invention may also contain an antistain agent. Suitable antistain agents include, for example, carboxylic acids such as those disclosed in U.S. Pat. No. 4,256,731, amino carboxylate compounds such as those disclosed in U.S. Pat. No. 4,080,441, phosphonoacetic acid, as disclosed in U.S. Pat. No. 4,118,474, or polyvinylpyrrolidone as disclosed in WO 93/16681. The antistain agent may be incorporated into the composition or may be provided as a separate composition, for use after the composition of the invention.

The pH of the formulation when diluted in the ratio of 3:1 with water should suitably be less than 10.0, for example from 5.5 to 9.0.

Suitably a composition according to the invention will have a viscosity of about 80,000 to about 500,000 cps at 25° C. which is necessary for producing a product that is comparable to conventional oral care compositions that have consumer acceptability. The viscosity of the oral care composition may be measured using a TF 20 spindle Brookfield Viscometer.

The present invention also provides a method of combating dental erosion and/or tooth wear which comprises applying an effective amount of a composition as hereinbefore defined to an individual in need thereof.

The present invention also provides a method of combating dental and/or root caries which comprises applying an effective amount of a composition as hereinbefore defined to an individual in need thereof.

The present invention also provides a method of combating dentine hypersensitivity which comprises applying an effective amount of a composition as hereinbefore defined to an individual in need thereof.

The following Examples illustrate the invention.

### EXAMPLES

### Example 1—Toothpaste Formulations

% w/w

| | Formulation | Formulation |
|---|---|---|
| Ingredient Name | I | II |
| Glycerol | 85.074 | 85.074 |
| Natrosol MX (HEC) | 1.100 | 1.100 |
| Aerosil 300 | 3.200 | 3.200 |
| Hydrated silica | — | — |
| PEG-8 | — | — |
| Calcium Sodium Phosphosilicate | 5.000 | 5.000 |
| Sodium Lauryl Sulphate | 1.100 | — |
| Sodium Fluoride | 0.315 | 0.315 |
| Titanium Dioxide | 1.000 | 1.000 |
| Flavour Oil 1 | 1.030 | — |
| Cocamidopropyl Betaine | — | 1.200 |
| Sodium Methyl Cocoyl Taurate | — | 1.200 |
| Carbomer (polyacrylic acid) | — | — |
| Saccharin Sodium | 0.350 | 0.350 |
| Flavour Oil 2 | — | 1.030 |
| Total | 100 | 100 |

Formulation III—used for comparative purposes (not a composition of the invention)

Formulation III is a commercially available control formulation comprising a calcium sodium phosphosilicate and a polyacrylic acid. Formulation III does not comprise a hydroxyethyl cellulose polymer or a pyrogenic silica, but is otherwise similar to Formulations I and II.

Formulations I and II above were prepared according to the following process: Using a suitable vessel, HEC and glycerine were stirred together and heated to a temperature of at least 80° C., but no higher than about 110° C., to form a clear mixture. The heating was then stopped and the mixture was allowed to cool naturally to room temperature. As the mixture was cooling down, the Aerosil was dispersed into the mixture using a high shear mixer such as an IKA 250 Ultra-Tirrax Disperser Homogenizer, and a clear gel was formed. All other ingredients of the formulation, with the exception of the flavour oil, were dispersed in the gel, at high shear, to produce a homogenous gel mixture. The flavour oil component was added once the mixture had cooled to a temperature lower than 40° C. Whilst some gel-like properties were lost during the last two steps of the manufacturing process resulting in a temporary drop in viscosity, gel structure was rebuilt within a few hours as a result of the thixotropic nature of the mixture.

### Example 2—Determination of Kinetics of Layer Formation Using a Modified QCMD

### Introduction

A novel technique was developed for measurement of mass deposition of material from a calcium- and phosphate-containing dentifrice using a Quartz Crystal Microbalance (QCM). The QCM is a nanogram-sensitive instrument that allowed the measurement of relative mass changes on the surface of a quartz crystal under the influence of an oscillating electric field using the piezoelectric effect (Dixon, M. C. Quartz Crystal Microbalance with Dissipation Monitoring: Enabling Real-Time Characterization of Biological Materials and Their Interactions. Journal of Biomolecular Techniques. 19, 2008, Vol. 3.). The QCM with Dissipation Model Q-Sense E1, manufactured by Biolin Scientific AB was used in the present study. The QCM is a modular system designed primarily for use with liquid samples. The different modules can provide flow or in one case a static no flow open module. To deposit material onto a sensor in the flow system, the sample must be pumped through. This does not provide control on how material is deposited since the sample has to go through piping and then to finally underflow deposit onto a surface.

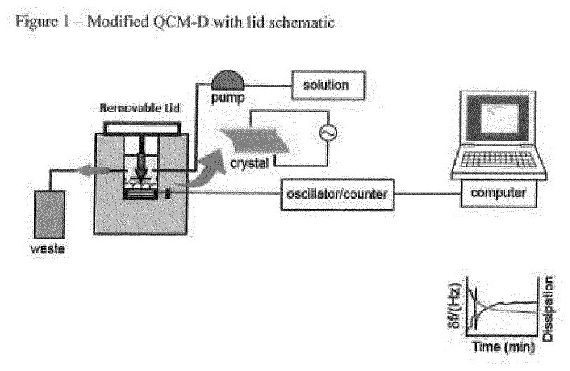

To overcome this issue a modified flow device was designed that had a removable lid as shown FIG. 1. This allowed for material to be precisely deposited onto the surface of the sensor, in this case with a pipette. It was necessary to replace the lid back onto the device otherwise atmospheric movement above the sensor could have been measured, inadvertently.

Preparation of Artificial Saliva (AS)

Artificial saliva was prepared by mixing the ingredients shown in Table 2. KOH was used to reduce the pH to 7.

TABLE 2

| | | |
|---|---|---|
| Solu | Mols dm⁻³ | g/L |

| | | | |
|---|---|---|---|
| Magnesium Chloride | 0.2 | mM | 0.01904 |
| Calcium chloride di-hydrate | 1.0 | mM | 0.14702 |
| Potassium di-hydrogen | 4.0 | mM | 0.54436 |
| orthophosphate | | | |
| HEPES (N-2Hydroxyethylpiperazine- | 20 | mM | 4.766 |
| N′-ethanethesulphonic acid) | | | |
| Potassium chloride | 16.0 | mM | 1.1928 |
| Ammonium chloride | 4.5 | mM | 0.2407 |

Test Samples

The following test samples were used in the study:
1. Example 3—Formulation III—(Comparative formulation) (1:3 (paste: AS) slurry)
2. Example 1—Formulation I (1:3 (paste: AS) slurry)
3. Example 2—Formulation II (1:3 (paste: AS) slurry)
4. Bioactive glass powder D50 of about 5.0 microns 1.25% by weight suspension in Artificial Saliva
5. Bioactive glass powder D50 of about 14 micron 1.25% by weight suspension in Artificial Saliva (equivalent amount of Bioactive Glass to above slurry)

Methodology

A QCM Hydroxyapatite-coated quartz crystal from Biolin Scientific Ab was inspected for any defects and cleaned with air and then subjected to UV/Ozone cleaning for 10 mins with a UV/Ozone cleaner such as with the UVC-1014 cleaner available from NanoBioanyltics, Max-Planck-Str. 3, 12489 Berlin, Germany. The crystal was then placed within the QCM instrument in the correct orientation as directed by the manufacturer and the instrument's guiding points, with the modified block and lid sealed. Artificial saliva was flowed over the sensor at a rate of 300 μL/min until a stable signal was achieved—The test sample and AS were weighed and prepared with the following weights: 2 g of paste and 60 mL of AS. The AS was pipetted into a beaker prior to each baseline measurement, during the 15 mins baseline record. After 15 minutes of baseline recording, the measurement was stopped and then restarted again. The flow of AS was then recorded for an additional 3 minutes and then stopped. The cover was then unscrewed and the paste and AS were vigorously mixed for 20 seconds to form a test suspension. Immediately after mixing 400 μl of the test suspension was pipetted into the QCM cell. The test suspension was in contact with the HA crystal for 2 minutes, no flow was applied. An AS flush was then applied to the crystal inside the cell, i.e. the AS speed was increased from the original 300 to 400 μL/min for 30 seconds. After flushing, the flow of AS at a rate of 300 μL/min was recorded for 2 hours. After measurement completion, the crystal was taken out of the QCM cell, gently rinsed with acetone and dried with argon.

Results

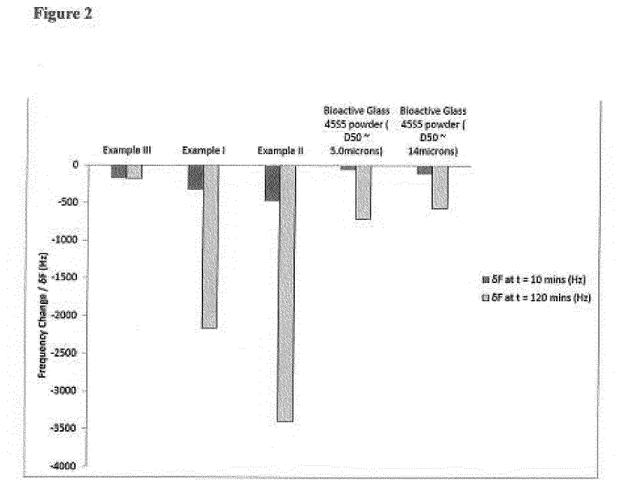

The results are demonstrated in FIG. 2.

Deposition of precipitate from the bioactive glass powders started to occur immediately and continued to occur during the two hour test period. Differences between the two powder samples at the two hour time point could be attributed to particle size differences between the two samples which would fit current theories on smaller particle sizes having a higher reactivity. Deposition of precipitate from the commercially available paste (Formulation III) was observed initially, but thereafter no significant deposition was observed. It appeared that deposition of material from the commercially available toothpaste was suppressed during the experiment. In contrast, a significant amount of precipitate deposition was observed with Formulations I and II. This could be due in part at least to pyrogenic silica being nucleating sites for HA formation, Aerosil is pyrogenic silica that is formed from sililic acid. Bioactive glass breaks down to sililic acid to allow the re-precipitation of calcium and phosphate that has been released on this sililic acid.

### Example 3—Hydraulic Conductance

### Introduction

Hydraulic conductance (Hc) is a methodology used to measure the extent of dentine tubule occlusion (Greenhill, Joel D., and David H. Pashley. “The effects of desensitizing agents on the hydraulic conductance of human dentin in vitro.” Journal of Dental Research 60.3 (1981): 686-698.). Hc was performed on three dentifrice formulations; Formulations I and II and Formulation III (a commercially available dentifrice formulation containing bioactive glass (comparative formulation)).

Methodology

Sound caries free human molars were sectioned and dentin discs extracted from between the crown and the pulp cavity (˜800 μm thick). These discs were then polished flat on both sides, initially with 800 grit paper, and then with 2500 grit paper (to a thickness of <500 μm). After polishing, the discs were placed into a 10% w/w citric acid solution and sonicated for 2 minutes. They were then rinsed under deionised water and subsequently soaked in deionised water for 10 minutes. 10 dentine discs were used for each dentifrice treatment in this experiment.

The hydraulic conductance equipment was connected to a compressed air supply and the solvent chamber pressurised to 1.0 PSI. A dentin disc was placed into the Pashley cell and Earles solution passed through the system. An air bubble was introduced into the capillary tube via the input port and allowed to proceed along the capillary tube for a few seconds before being timed from a defined start point. The starting position of the bubble was measured and the distance travelled over the following 5 minutes was measured at one minute intervals. Acceptance criteria for untreated dentin discs is defined as those having a hydrodynamic flow rate of 1.0-10.0 mm/min. Any dentine discs falling outside this range were considered to be inadequate for use in the Hc experiment.

Neat pastes were applied to the dentin discs using a Benda brush for 10 seconds. After treatment, the discs were soaked in the corresponding formulation for a further 2 minutes. The disks were then rinsed with deionised water and a second air bubble introduced into the capillary tube. After a brief pause to allow equilibration, the distance travelled by the bubble was again measured over five minutes, at one minute intervals. The reduction in flow between untreated and treated dentin was calculated. The Pashley cell was then removed from the hydraulic conductance equipment and placed into a 60 ml Sterilin jar containing ˜20 ml of artificial saliva. The Sterilin jar was then incubated at 37° C. for 24 hrs.

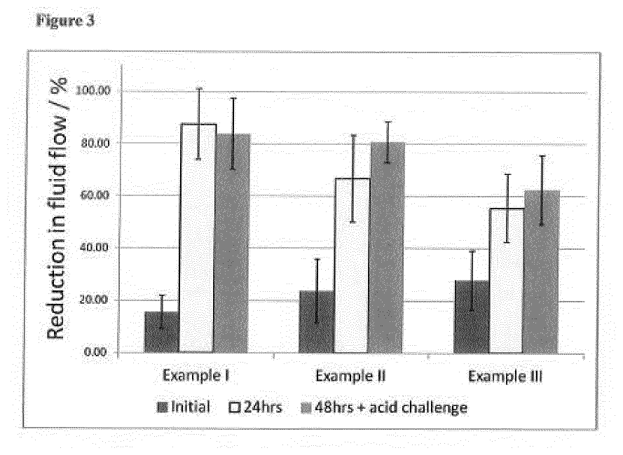

After 24 hrs incubation in artificial saliva, the cell was re-attached to the hydraulic conductance chamber and the hydrodynamic flow re-measured. The reduction in flow between untreated dentine discs and discs that had been treated for 24 hrs was calculated. A second treatment dentifrice was then performed as described above, followed by a further 24 hr incubation in artificial saliva. After this second incubation period, the cell and disc were again removed and rinsed with deionised water then placed into 50 ml of Coca Cola for 2 minutes. A final fluid flow measurement was performed as above. The results of this experiment are shown below (FIG. 3) as percentage reduction in fluid flow after treatment vs initial fluid flow before treatment.

Results

FIG. 3 shows the % reduction in fluid flow through dentine tubules after treatment with test dentifrices. Treatment with the commercially available dentifrice containing bioactive glass leads to a reduction in fluid flow through dentine tubules as expected at all time points. Treatment with Formulations I and II dentifrices lead to a reduction in fluid flow through dentine tubules to a statistically greater extent than the commercial dentifrice (Formulation III) after 24 and 48 hr treatment. This data suggests that compositions described herein would be effective and may even potentially offer improvements in the treatment of dentine hypersensitivity.

1. A non-aqueous oral care composition comprising a source of calcium ions, a source of phosphate ions, a humectant, a hydroxyethyl cellulose polymer and a pyrogenic silica.

2. A non-aqueous oral care composition according to claim 1 wherein the source of calcium ions and the source of phosphate ions is a calcium phosphate compound.

3. A non-aqueous oral care composition according to claim 2 wherein the calcium phosphate compound is selected from a bioactive glass, calcium glycerophosphate, dicalcium phosphate dihydrate, tetracalcium phosphate, octacalcium phosphate, amorphous calcium phosphate, apatite, α-tricalcium phosphate or a mixture thereof.

4. A non-aqueous oral care composition according to claim 3 wherein the calcium phosphate compound is a bioactive glass.

5. A non-aqueous oral care composition according to claim 4 wherein the bioactive glass is calcium sodium phosphosilicate.

6. A non-aqueous oral care composition according to claim 5 wherein the is formed from a combination of 40% to 60% by weight silicon dioxide, from 10% to 40% by weight calcium oxide, from 10% to 35% by weight sodium oxide, from 2% to 8% phosphorus oxide, from 0% to 25% by weight calcium fluoride, from 0% to 10% by weight boron oxide, from 0% to 8% by weight potassium oxide, from 0% to 5% magnesium oxide.

7. A non-aqueous oral care composition according to claim 5 wherein the calcium sodium phosphosilicate comprises about 45% by weight silicon dioxide, about 24.5% by weight sodium oxide, about 6% by weight phosphorus oxide, and about 24.5% by weight calcium oxide.

8. A non-aqueous oral care composition according to any one of claims 2 to 7 wherein the calcium phosphate compound is present in an amount ranging from 1 to 20% by weight of the composition.

9. A non-aqueous oral care composition according to claim 1 wherein the source of calcium ions is selected from calcium chloride, calcium bromide, calcium nitrate, calcium acetate, calcium gluconate, calcium benzoate, calcium glycerophosphate, calcium formate, calcium fumarate, calcium lactate, calcium butyrate and calcium isobutyrate, calcium malate, calcium maleate, calcium propionate, calcium carbonate, calcium silicate, calcium oxide, calcium sulphate, calcium alginate or mixtures thereof.

10. A non-aqueous oral care composition according to claim 9 wherein the source of calcium ions is present in an amount ranging from 1 to 20% by weight of the composition.

11. A non-aqueous oral care composition according to claim 1 wherein the source of phosphate ions is selected from sodium dihydrogen phosphate, disodium hydrogen phosphate, sodium pyrophosphate, tetrasodium pyrophosphate, sodium tripolyphosphate, sodium hexametaphosphate, potassium dihydrogenphosphate, trisodium phosphate, tripotassium phosphate or mixtures thereof.

12. A non-aqueous oral care composition according to claim 11 wherein the source of phosphate ions is present in an amount ranging form 1 to 20% by weight of the composition.

13. A non-aqueous oral care composition according to any one of claims 1 to 12 wherein the humectant is glycerine.

14. A non-aqueous oral care composition according to any one of claims 1 to 13 wherein the humectant is present in an amount ranging from 20 to 90% by weight of the composition.

15. A non-aqueous oral care composition according to any one of claims 1 to 14 wherein the hydroxyethyl cellulose polymer is Natrosol MX.

16. A non-aqueous oral care composition according to any one of claims 1 to 15 wherein the hydroxyethyl cellulose polymer is present in an amount ranging from 0.1 to 7.5% by weight of the composition.

17. A non-aqueous oral care composition according to any one of claims 1 to 16 wherein the pyrogenic silica is Aerosil 300.

18. A non-aqueous oral care composition according to any one of claims 1 to 17 wherein the pyrogenic silica is present in an amount ranging from 1 to 10% by weight of the composition.

19. A non-aqueous oral care composition according to any one of claims 1 to 18 comprising an ionic fluorine-containing compound.

20. A non-aqueous oral care composition according to any one of claims 1 to 19 for use in treating dentine hypersensitivity.
